(19) United States
(12) Patent Application Publication
(10) Pub. No.: US 2025/0145980 A1
(43) Pub. Date: May 8, 2025

(54) ClyRD, A NOVEL CHIMERIC ENDOLYSIN FROM CLOSTRIDIUM PERFRINGENS PHAGES WITH ENHANCED ANTIBACTERIAL ACTIVITY

(71) Applicant: FOUNDATION FOR RESEARCH AND BUSINESS, SEOUL NATIONAL UNIVERSITY OF SCIENCE AND TECHNOLOGY, Seoul (KR)

(72) Inventors: Min Suk KONG, Seoul (KR); Je Na KIM, Sejong-si (KR); Su Young SEO, Gyeonggi-do (KR)

(73) Assignee: FOUNDATION FOR RESEARCH AND BUSINESS, SEOUL NATIONAL UNIVERSITY OF SCIENCE AND TECHNOLOGY, Seoul (KR)

(21) Appl. No.: 18/934,525

(22) Filed: Nov. 1, 2024

(30) Foreign Application Priority Data
Nov. 3, 2023 (KR) 10-2023-0150738

(51) Int. Cl. C12N 9/80; G01N 33/569

(52) U.S. Cl. CPC C12N 9/80; G01N 33/56911; C07K 2319/60; C12Y 305/01004

(57) ABSTRACT

A chimeric endolysin ClyRD of the present disclosure has antibacterial activity against bacteria that may cause foodborne diseases. Therefore, the chimeric endolysin ClyRD may be used as a natural controlling agent, a therapeutic agent or a preventive agent in the food industry.

### BACKGROUND

### Sequence Listing

A sequence listing in electronic (XML file) format is filed with this application and incorporated herein by reference. The name of the XML file is “Sequence_Listing-1638A.xml”; the file was created on Nov. 1, 2024; the size of the file is 22,230 bytes.

### 1. TECHNICAL FIELD

This application claims the benefit of the filing date of Korean Patent Application No. 10-2023-0150738 filed with the Korean Intellectual Property Office on Nov. 3, 2023, the entire contents of which are incorporated herein by reference.

The work in the present disclosure was supported by the individual basic research program through the National Research Foundation of Korea (NRF) funded by the Korean government (the Ministry of Science and ICT) from March 2020 to February 2023 (Project Number: 2020R1C1C1008127).

The work in the present disclosure was also supported by the Korea Health Technology R&D Project through the Korea Health Industry Development Institute (KHIDI), funded by the Ministry of Health and Welfare of the Republic of Korea (Project Number: RS-2023-KH136866).

The present disclosure relates to discovering a more effective material for control and detection of Clostridium perfringens (C. perfringens) by isolating C. perfringens bacteriophages directly from an environmental sample and identifying and swapping several domains of a novel endolysin obtained through genomic analysis.

### 2. RELATED ART

Clostridium perfringens is a gram-positive, facultative anaerobic bacterium that is widely found in raw meat, poultry, the intestinal tract of animals, and the natural environment, and is characterized by forming heat-resistant spores. Clostridium perfringens is the second most common cause of foodborne illness in most developed countries and is a zoonotic pathogen that causes symptoms, such as diarrhea, abdominal pain, and gas gangrene, when consumed. Heat-resistant spores of Clostridium perfringens are not killed even during the cooking process, but rather germinate into vegetative cells and grow in the food. When a large number of vegetative cells were ingested, cells reach the intestines and release toxins while sporulation, causing symptoms of food poisoning. In order to preemptively detect and prevent food contamination with Clostridium perfringens and its spores, there is an urgent need to develop a technology for rapidly and accurately controlling them. Bacteriophages are viruses with host specificity, and have the great advantage of killing only the target host, unlike antibiotics that kill all bacteria, including beneficial bacteria. Not only bacteriophages, but also proteins produced by phages may be used as means for detecting and controlling bacteria, and representative examples thereof include endolysins that are antibacterial proteins. Endolysins are enzymes used by bacteriophages to degrade the cell wall of host bacteria after they infect the bacteria and multiply inside the cell, allowing the progeny phages to escape from the bacteria. In general, a protein derived from the bacteriophage shows a broader antibacterial spectrum than the parent phage. Gram-positive phage endolysins mainly have two domains: an enzymatically active domain (EAD) that hydrolyzes the peptidoglycan of the host bacterial cell wall, and a cell wall-binding domain (CBD) that binds to a specific portion of the host bacterial cell wall, with a flexible linker between the two domains. Among these domains, the CBD has the ability to strongly adhere to the host bacteria and also has high host specificity, and thus it may be used as a detection medium.

Currently commercialized rapid detection methods for bacteria mainly use antibodies, but have disadvantages in that the production of antibodies requires a lot of time and economic cost and there are concerns about the occurrence of non-specific binding. In addition, these methods have a disadvantage in that stability is low due to the significant influence of external factors such as temperature, salt concentration, and pH.

Although the CBD can be considered as a detection medium that can replace antibodies, the CBD has the limitation of not being able to bind to Clostridium perfringens spores, which are known to be abundant in nature. Endolysins have also received attention as next-generation antibacterial agents, but due to the nature of the proteins, they are often unstable depending on temperature, pH, or salt concentration, and often have low activity in foods. Therefore, there is an urgent need to develop a more reliable antibacterial agent for controlling Clostridium perfringens present in foods.

### SUMMARY

The CBD has received attention for its potential as a detection material for live target microorganisms, but has limitations in detecting microorganisms that exist in the form of spores. The inventors of the present disclosure analyzed the genomes of Clostridium perfringens phages Reka1 and Dolk21, which were directly isolated from nature, and conducted studies on the domains of endolysins PlyReka1 and PlyDolk21 from each phage. The inventors have found that, in PlyReka1, there are an enzymatic active domain (EAD) with high host specificity and a spore-binding domain (SBD) that specifically binds to Clostridium perfringens spores, and in PlyDolk21, there is a cell wall-binding domain (CBD) that binds only to the cell wall of the target host bacteria.

The inventors have found that PlyReka1 exhibits specific lytic activity against the host, but does not exhibit lytic activity in foods artificially contaminated with the host bacteria. The inventors could find that a novel 1 endolysin produced by linking the EAD domain responsible for the excellent lytic activity of PlyReka1 with the CBD of PlyDolk21 instead of the existing SBD exhibited activity similar to that of the parent phage endolysin PlyReka1, and at the same time, was more stable under higher salt concentration and pH conditions and could kill only the host bacteria in actual foods.

The SBD obtained in the present disclosure may be used in various ways for the detection and study of Clostridium perfringens spores, and the novel endolysin obtained by combining domains obtained from various endolysins shows its superiority in that it can exhibit better activity and stability than the parent phage. Thereby, it has been found that the novel endolysin may be used as a probe for diagnosis and detection of live Clostridium perfringens and spores present in nature.

The present disclosure relates to a method of producing a chimeric endolysin protein by isolating Clostridium perfringens bacteriophages Reka1 and Dolk21, analyzing the genome of each of the bacteriophages, and then using a cell wall binding-domain (CBD) and a spore-binding domain (SBD), which are the features of the bacteriophages.

The chimeric endolysin is hereinafter referred to as ClyRD.

In one embodiment of the present disclosure, the chimeric endolysin protein comprises an enzymatically active domain (EAD), a spore-binding domain (SBD), and a cell wall-binding domain (CBD).

The protein is one in which the enzymatically active domain, the spore-binding domain, and the cell wall-binding domain are sequentially linked together, but the order of the linking is not limited thereto.

The enzymatically active domain, the spore-binding domain, and the cell wall-binding domain are derived from the endolysin proteins of bacteriophages.

The bacteriophages are bacteriophages Reka1 and Dolk21, and the endolysin proteins derived from the bacteriophages are PlyReka1 and PlyDolk21, respectively.

The chimeric endolysin protein may be contained in a composition for detecting bacteria that cause foodborne disease or for preventing or treating the foodborne disease.

The composition containing the chimeric endolysin protein serves to inhibit the growth and proliferation of bacteria that cause foodborne disease.

The chimeric endolysin protein has antibacterial activity against a Clostridium sp. strain.

The bacteriophages are Clostridium perfringens bacteriophages, and the Clostridium sp. strain is Clostridium perfringens.

The Clostridium perfringens is a bacterium that causes foodborne disease and zoonotic infectious disease.

The foodborne disease refers to a variety of foodborne infections caused by pathogens such as bacteria that contaminate food, and more than 250 foodborne diseases have been identified. Main bacteria known to cause foodborne diseases include norovirus, Salmonella, Clostridium perfringens, and Staphylococcus aureus.

In one embodiment of the present disclosure, Reka1 may be a phage of the Podoviridae family, and Dolk21 may be a phage of the Myoviridae family.

The chimeric endolysin ClyRD of the present disclosure may demonstrate the potential for development of chimeric endolysins, and may be used as a natural controlling agent in the food industry and as an effective therapeutic and preventive agent against bacteria such as spores.

### BRIEF DESCRIPTION OF THE DRAWINGS

FIG. 1 shows transmission electron microscopy images of bacteriophages Reka1 (A) and Dolk21 (B), respectively.

FIG. 2 is a genome map of the Reka1 phage.

FIG. 3 is a genome map of the Dolk21 phage.

FIG. 4 shows the results of comparing the amino acid sequences between PlyReka1 and other Clostridium phage endolysins.

FIG. 5 shows the results of comparing the amino acid sequences between the endolysin PlyDolk21 of the Dolk21 phage and endolysins derived from other Clostridium perfringens bacteriophages.

FIG. 6 shows the results of analyzing the 3D structure of PlyDolk21 using the Pymol program. In FIG. 6, the red color represents the Amidase_2 domain, the purple color represents the SH3_3 domain, and the yellow portion is a coil predicted as a portion that can be cut into the putative CBD.

FIG. 7 is a schematic diagram showing the domains and putative CBD region in PlyDolk21.

FIG. 8 shows the results of analyzing EGFP::PlyDolk21_CBD protein (size: 56 kDa) by SDS-PAGE after purification using Ni-NTA chromatography.

FIG. 9 shows the results of analyzing the binding activity of EGFP::PlyDolk21_CBD. (A): bright field image, (B): fluorescence image, (C): merged images, (D): fluorescence image of other Gram-positive bacteria, and (E): fluorescence image of Gram-negative bacteria.

FIG. 10 schematically shows PlyReka1 and EGFP-tagged PlyReka1_SPOR.

FIG. 11 shows the results of SDS-PAGE of EGFP-tagged PlyReka1_SPOR protein (size: 38.92 kDa) purified using Ni-NTA chromatography.

FIG. 12 depicts fluorescence microscope images showing the binding of PlyReka1_SPOR to Clostridium perfringens vegetative cells and spores.

FIG. 13 shows the results of SDS-PAGE of purified PlyReka1 protein (27.38 kDa) and a quantitative graph showing the lytic activity of the protein.

FIG. 14 schematically shows overlapping PCR.

FIG. 15 shows the predicted 3D protein structure of ClyRD.

FIG. 16 shows the results of SDS-PAGE of ClyRD protein (size: 49.42 kDa) purified using Ni-NTA chromatography and a graph showing the lytic activity of the protein.

FIG. 17 depicts graphs showing the results of analyzing the activity of endolysins in actual foods. (A): sterilized milk, and (B): bone broth.

FIG. 18 depicts graphs showing the results of optimizing the pH, salt concentration and temperature conditions of ClyRD compared to the parent endolysin.

### DETAILED DESCRIPTION

The present disclosure relates to a method of producing a ClyRD endolysin protein by isolating Clostridium perfringens bacteriophages Reka1 and Dolk21, analyzing the genome of each of the bacteriophages, and then using a cell wall binding-domain (CBD) and a spore-binding domain (SBD), which are the features of the bacteriophages.

In one embodiment of the present disclosure, Reka1 may be a phage of the Podoviridae family, and Dolk21 may be a phage of the Myoviridae family.

In one embodiment of the present disclosure, the endolysin from Clostridium perfringens phage Reka1 is the 12th ORF among 22 ORFs of the phage genome.

In one embodiment of the present disclosure, the 12th ORF of the endolysin from Clostridium perfringens phage Reka1 is PlyReka1.

In one embodiment of the present disclosure, the PlyReka1 may have N-acetylmuramoyl-L-alanine amidase at the N-terminus.

In one embodiment of the present disclosure, the endolysin from Clostridium perfringens phage Dolk21 is the 27th ORF among 71 ORFs of the phage genome.

In one embodiment of the present disclosure, the 27th ORF of the endolysin from Clostridium perfringens phage Dolk21 is PlyDolk21.

In one embodiment of the present disclosure, the Clostridium perfringens phage PlyDolk21 has a cell wall-binding domain (CBD).

The PlyDolk21 may be translated from the DNA sequence of SEQ ID NO: 9.

The PlyDolk21 may consist of the amino acid sequence of SEQ ID NO: 10.

The PlyDolk21_CBD (CBD domain) may be translated from the DNA sequence of SEQ ID NO: 11.

The PlyDolk21_CBD (CBD domain) may consist of the amino acid sequence of SEQ ID NO: 12.

In one embodiment of the present disclosure, the CBD of PlyDolk21 consists of amino acid residues 157 to 388, specifically amino acid residues 300 to 387, more specifically amino acid residues 335 to 386, in the amino acid sequence of PlyDolk21.

In one embodiment of the present disclosure, amino acid residues 335 to 386 in the amino acid sequence of PlyDolk21 correspond to an SH3-3 domain, which is a CBD domain.

In one embodiment of the present disclosure, the size of the PlyDolk21-CBD protein is 48 to 63 kDa, specifically 52 to 59 kDa, more specifically 56 kDa.

In one embodiment of the present disclosure, the concentration of the PlyDolk21-CBD protein is 4 to 7 mg/ml, specifically 4.5 to 6.5 mg/ml, more specifically 5.8 mg/ml.

In one embodiment of the present disclosure, the PlyDolk21-CBD protein may specifically bind to Clostridium perfringens. More specifically, the PlyDolk21-CBD protein may not exhibit binding activity to Gram-positive bacteria or Gram-negative bacteria other than Clostridium perfringens.

In one embodiment of the present disclosure, the PlyDolk21 CBD protein may bind to the septal region and pole of a Clostridium perfringens strain.

In one embodiment of the present disclosure, the PlyReka1 may have an amidase-3 domain at the N-terminus.

In one embodiment of the present disclosure, the C-terminus of the PlyReka1 may have a spore-binding domain (SBD).

The PlyReka1 may be translated from the DNA sequence of SEQ ID NO: 3.

The PlyReka1 may consist of the amino acid sequence of SEQ ID NO: 4.

The PlyReka1_SPOR (SBD domain) may be translated from the DNA sequence of SEQ ID NO: 5.

The PlyReka1_SPOR (SBD domain) may consist of the amino acid sequence of SEQ ID NO: 6.

In one embodiment of the present disclosure, the SBD domain of the PlyReka1 is a domain consisting of amino acid residues 131 to 212 in the amino acid sequence of the PlyReka1.

In one embodiment of the present disclosure, the size of the PlyReka1 SBD protein is 20 to 35 kDa, specifically 25 to 30 kDa, more specifically 27.38 kDa.

The method for identifying the PlyDolk21_CBD and the PlyReka1 SBD was performed using a vector containing enhanced green fluorescent protein (EGFP).

The EGFP-PlyDolk21_CBD containing EGFP may be translated from the DNA sequence of SEQ ID NO: 13.

The EGFP-PlyDolk21_CBD containing EGFP may consist of the amino acid sequence of SEQ ID NO: 14.

The EGFP-PlyReka1 SBD containing EGFP may be translated from the DNA sequence of SEQ ID NO: 7.

The EGFP-PlyReka1_SBD containing EGFP may consist of the amino acid sequence of SEQ ID NO: 8.

In the present disclosure, a novel chimeric endolysin, ClyRD, was produced by linking amidase 3, an enzymatically active domain of PlyReka1, with the CBD of PlyDolk21, which has been confirmed to have excellent binding ability.

In one embodiment of the present disclosure, the ClyRD may be a chimeric endolysin obtained by linking the CBD of PlyDolk21 with the EAD of PlyReka1 by overlapping PCR.

In one embodiment of the present disclosure, the ClyRD may have antibacterial activity against bacteria that cause food poisoning. Specifically, the ClyRD may have antibacterial activity against Gram-positive bacteria. More specifically, the ClyRD may have antibacterial activity against Clostridium perfringens.

The ClyRD may have the function of EAD, CBD or SBD. Specifically, the ClyRD may have EAD, CBD and SBD.

The EAD of the ClyRD may consist of amino acid residues 0 to 131 in the amino acid sequence of SEQ ID NO: 2.

The SBD of the ClyRD may consist of amino acid residues 132 to 212 in the amino acid sequence of SEQ ID NO: 2.

The CBD of the ClyRD may consist of amino acid residues 213 to 441 in the amino acid sequence of SEQ ID NO: 2.

In one embodiment of the present disclosure, the ClyRD may have a size of 35 to 75 kDa, specifically 48 to 63 kDa, more specifically 49.42 kDa.

The ClyRD may be translated from the DNA sequence of SEQ ID NO: 1.

The CLyRD may consist of the amino acid sequence of SEQ ID NO: 2.

The chimeric endolysin protein may be contained in a composition for detecting bacteria that cause foodborne disease or for preventing or treating the foodborne disease. The composition containing the chimeric endolysin protein serves to inhibit the growth and proliferation of bacteria that cause foodborne disease.

The chimeric endolysin protein has antibacterial activity against a Clostridium sp. strain.

The bacteriophages are Clostridium perfringens bacteriophages, and the Clostridium sp. strain is Clostridium perfringens.

Hereinafter, one or more embodiments will be described in more detail by way of examples. However, these examples are only to illustrate one or more embodiments, and the scope of the present disclosure is not limited to these examples.

### Example 1. Isolation of Bacteriophages and PlyReka1 Identification by Genomic Analysis

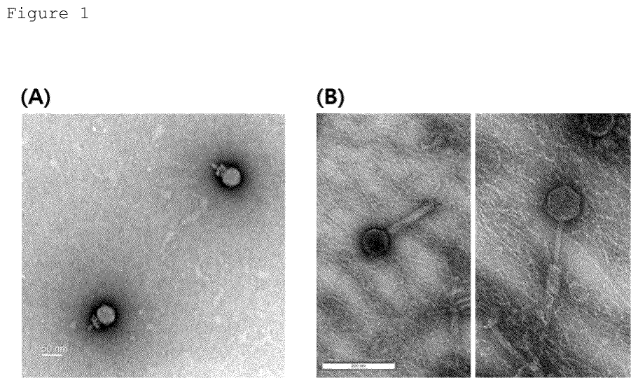

Bacteriophages Reka1 and Dolk21, which specifically infect Clostridium perfringens (C. perfringens), were isolated from the soil samples of Guri Wastewater Treatment Plant (Korea) and the lawn in Bucheon city, Korea, respectively. As a result of observing the morphology of the isolated Clostridium perfringens phages Reka1 and Dolk21 by TEM, it was confirmed that Reka1 is a phage of the Podoviridae family, and Dolk21 is a phage of the Myoviridae family (FIGS. 1A and 1B).

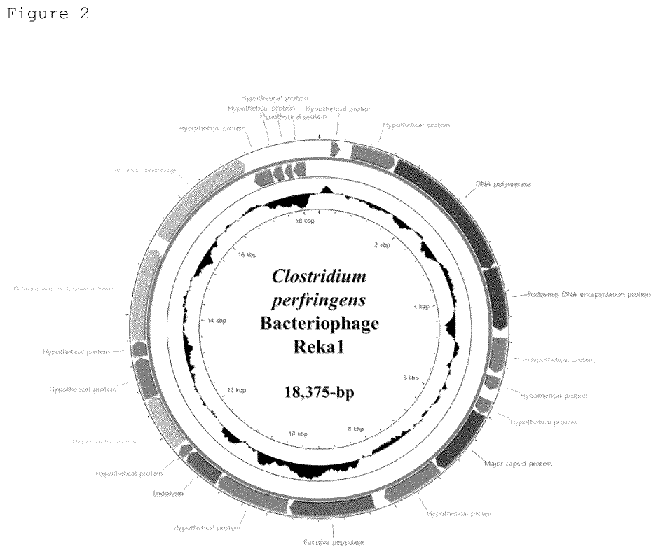

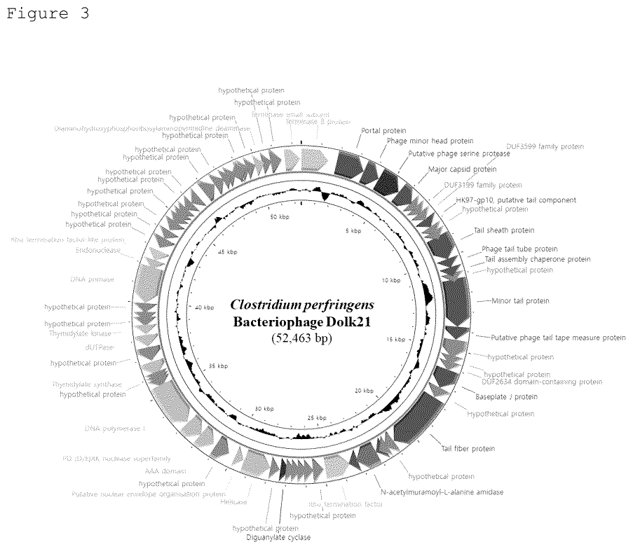

As a result of analyzing the genomes of the phages, it was confirmed that Reka1 was 18, 375 bp in length and Dolk21 was 52,463 bp in length. In addition, the results of genetic analysis of the phages showed that Reka1 had 22 ORFs and Dolk21 had 71 ORFs (FIGS. 2 and 3).

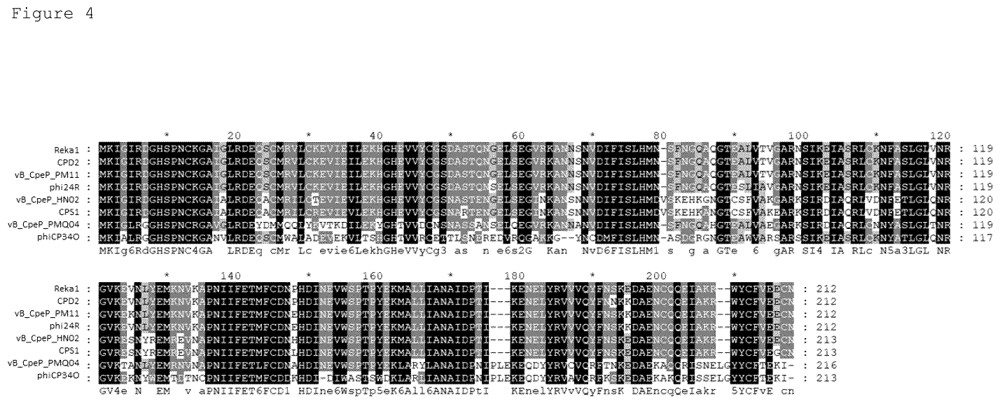

The 12^{th} ORF among the ORFs of the Reka1 phage was predicted to be N-acetylmuramoyl-L-alanine amidase, and thus was named the endolysin PlyReka1 from Reka1. As a result of amino acid sequence comparison, PlyReka1 showed 99% amino acid homology with the endolysin from Clostridium bacteriophage CPD2 (FIG. 4).

### Example 2. Prediction of CBD by Genomic Analysis of C. perfringens Endolysin PlyDolk21

The 27^{th} ORF of Dolk21, which was identified as an endolysin through lytic activity analysis, was named PlyDolk21, and the amino acid sequence of the cell wall-binding domain (CBD) of PlyDolk21 was analyzed using BlastP and InterProScan.

It was confirmed that there was an SH3_3 domain, a putative CBD, in the region 335-386 in the amino acid sequence of PlyDolk21, and the downstream portion including the SH3_3 domain had a very high similarity to the CBD of C. perfringens endolysin LysCPAS15, which had been previously studied for the CBD (FIG. 5).

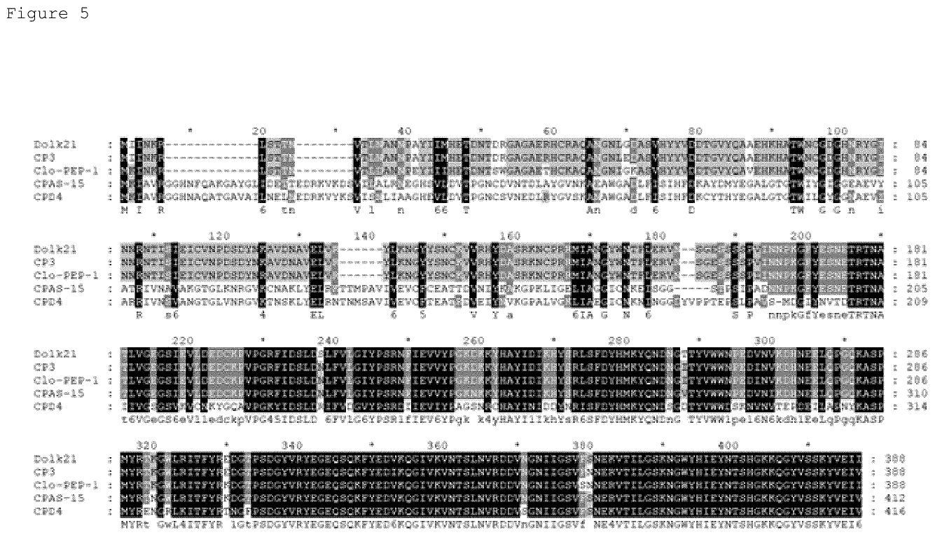

Based on the analysis of the protein secondary and tertiary structures (FIG. 6) predicted with reference to the results of the study (Jae-Hyun Cho et al.), amino acid residues 157 to 388 of PlyDolk21 were estimated to be a CBD and cloned.

### Example 3. Expression and Purification of PlyDolk21_CBD

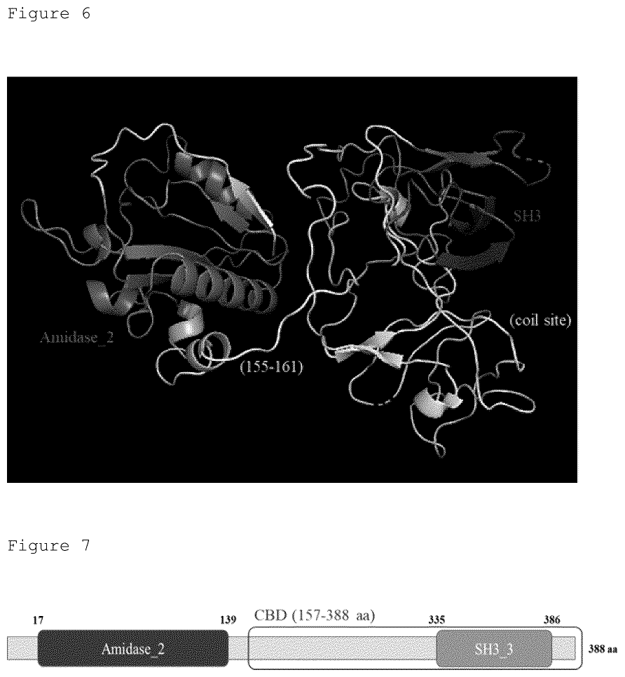

As a result of amplifying the putative CBD region from the endolysin PlyDolk21 plasmid by PCR, very high concentration of a DNA fragment could be obtained, and the fragment was confirmed to be of the expected size (711 bp, including the restriction enzyme sites) (FIG. 7). The amplified PCR product was purified using the MiniBEST DNA fragment purification kit (TakaRa). Next, the purified PlyDolk21_CBD and the pET28a::EGFP vector were digested with BamHI and HindIII and ligated together, and the ligation product was transformed into E. coli DH10B competent cells. It was confirmed that the resulting recombinant plasmid was expressed normally. For protein expression, the cloned plasmid was transformed into E. coli BL21 (DE3) competent cells, and the colony grown overnight was inoculated at 1/100 into LB medium containing kanamycin and grown at 37° C. and 250 rpm until the OD₆₀₀ value reached 0.6 to 0.8. Then, 0.5 mM IPTG was added thereto, and the bacterial cells were cultured at 18° C. and 250 rpm for 20 hours.

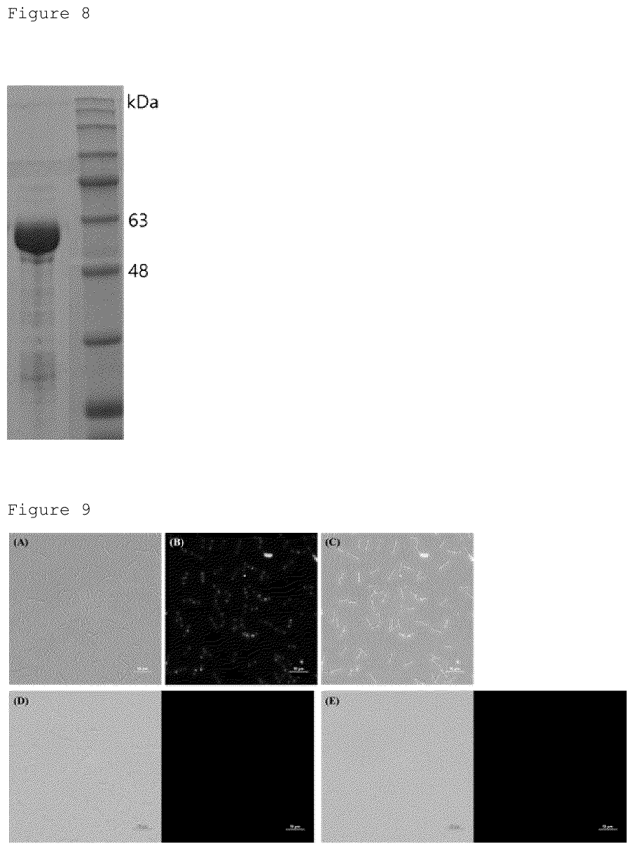

The cultured bacterial cells were precipitated by centrifugation (at 4,000×g and 4° C. for 15 min), the pellet was suspended in 5 mL of lysis buffer (50 mM Tris-Cl (pH 8.0), 200 mM NaCl), and the cells were lysed by sonication for 6 minutes using time intervals of 6 sec on and 4 sec off. After lysis, undissolved protein was removed by centrifugation (21, 000 ×g and 4° C. for 1 hour), and then 5 mM imidazole was added to the endolysin mixed with other proteins, and the mixture was mixed with Ni-NTA resin at 4° C. for 1 hour. Proteins not bound to the resin were removed by passage through a Poly-prep chromatography column (Biorad). In addition, non-specifically bound proteins were removed by sequentially passing lysis buffers containing 10 mM and 20 mM imidazole through the column, and finally, EGFP::PlyDolk21_CBD protein expected to be bound to the resin was eluted by passing lysis buffer containing 200 mM imidazole. The concentration of the obtained protein (56 kDa) was analyzed by Bradford assay, and the purity was analyzed by SDS-PAGE (FIG. 8).

The endolysin with high concentration and purity was subjected to buffer exchange from storage buffer to lysis buffer using a Zeba spin column, and finally the protein with a high concentration of about 5.8 mg/ml was stored at −80° C.

### Example 4. Confirmation of Binding of PlyDolk21_CBD to C. perfringens

Since the vector contained enhanced green fluorescent protein (EGFP) together with PlyDolk21_CBD, whether the CBD would actually bind to the cell wall was determined by confirming the green fluorescence image through EGFP binding assay. First, the host strain C. perfringens ATCC 13124 was grown until the OD₆₀₀ value reached 0.6 to 0.8, and then 1 ml of the bacterial cells were precipitated by centrifugation (at 21,000 ×g and 4° C. for 1 min) and washed twice with 1 mL of PBS buffer. Then, the pellet was suspended in 1 mL of PBS buffer, and 100 μL of the suspension was taken and mixed with 100 μL of a solution containing 2 μM EGFP::PlyDolk21 CBD in 1 mL of PBS buffer. The mixture of the bacterial cells and the protein (final protein concentration: 1 μM) was incubated at room temperature for 5 minutes. After the incubation, the bacterial cells were precipitated by centrifugation (at 21,000 ×g and 4° C. for 1 min) and washed twice with 200 μL of PBS to remove residual EGFP::PlyDolk21 CBD not bound to the cells and non-specifically bound proteins. Thereafter, the cells were resuspended in 10 μL of PBS, thereby preparing samples. Whether each sample would emit green fluorescence was observed using an inverted fluorescence microscope (ECLIPSE Ti2-E, Nikon) under the following conditions: DIA: 17.1, intensilight: ND 2, auto exposure: 1 second, and analog gain: 1.0×. As a result, it was shown that PlyDolk21_CBD bound to the cell wall, mainly in the septal region and pole (FIG. 9).

Host range analysis was performed by conducting the same experimental method on other C. perfringens strains and Gram-positive and Gram-negative bacteria (Table 1).

TABLE 1
Binding spectrum of PlyDolk21_CBD

| Species | Strain No. | PlyDolk21_CBD |
|---|---|---|
| C. perfringens | 2585 | + |
| C. perfringens | 2589 | + |
| C. perfringens | ATCC 3624 | + |
| C. perfringens | ATCC 13124 | + |
| C. perfringens | H3 | + |
| C. perfringens | H9 | + |
| C. perfringens | FD1 | + |
| Other Gram-positive | | |
| Bacillus cereus | ATCC 10987 | − |
| Bacillus subtilis | ATCC 23857 | − |
| Staphylococcus aureus | Newman | − |
| Listeria monocytogenes | ATCC 15313 | − |
| Geobacillus stearothermophilus | ATCC 10149 | − |
| Gram-negative | | |
| E. coli O157:H7 | ATCC 35150 | − |
| Salmonella Typhimurium | LT2 | − |
| Pseudomonas aeruginosa | PAO1 | − |
| Cronobacter sakazakii | ATCC 29544 | − |

+, activity of cell wall binding assay;
−, no activity

As a result of examining the binding spectrum, it was shown that the PlyDolk21_CBD protein bound to the cell walls of strains belonging to the same Clostridium perfringens species, and did not show the activity of binding to the remaining Gram-positive or Gram-negative bacteria.

This demonstrates that PlyDolk21_CBD is capable of binding very specifically to Clostridium perfringens. In particular, it was confirmed that PlyDolk21_CBD bound strongly to the septal region and pole.

### Example 5. Identification of Spore-Binding Domain (SBD) of PlyReka1

The results of domain analysis using Interproscan and BLASTP databases indicated that PlyReka1 has only one domain, which is amidase_3 (PF01520) at the N-terminus.

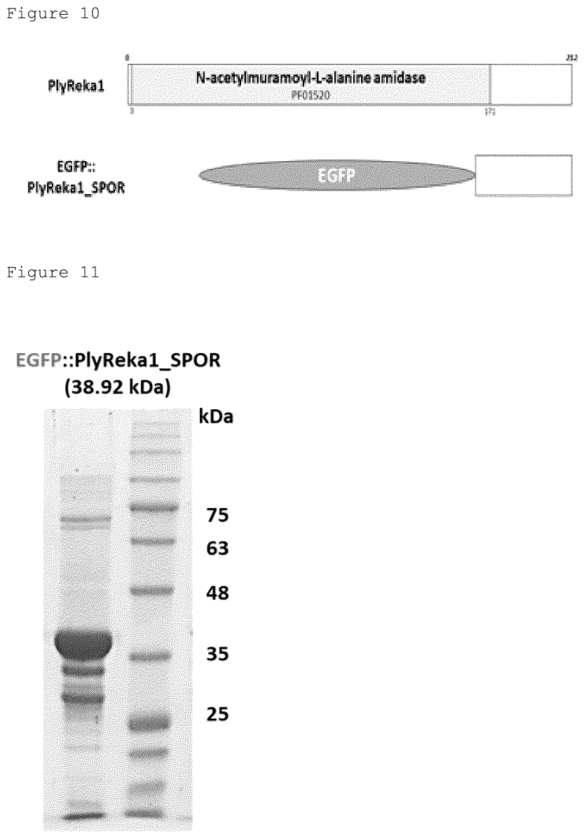

As a result of domain analysis, it was confirmed that, unlike the endolysins of general bacteriophages that use Gram-positive bacteria as host cells, in which the endolysins have an enzymatically active domain (EAD) and a cell wall-binding domain (CBD) at the N-terminus and C-terminus, respectively, PlyReka1 has an amidase-3 domain at the N-terminus, but has no predicted domain at the C-terminus (FIG. 10).

Therefore, the C-terminus (131-212 aa) of PlyReka1 was named PlyReka1_SPOR, and it was tagged with enhanced green fluorescent protein and (EGFP) subjected to E. coli overexpression and protein purification steps (FIG. 11).

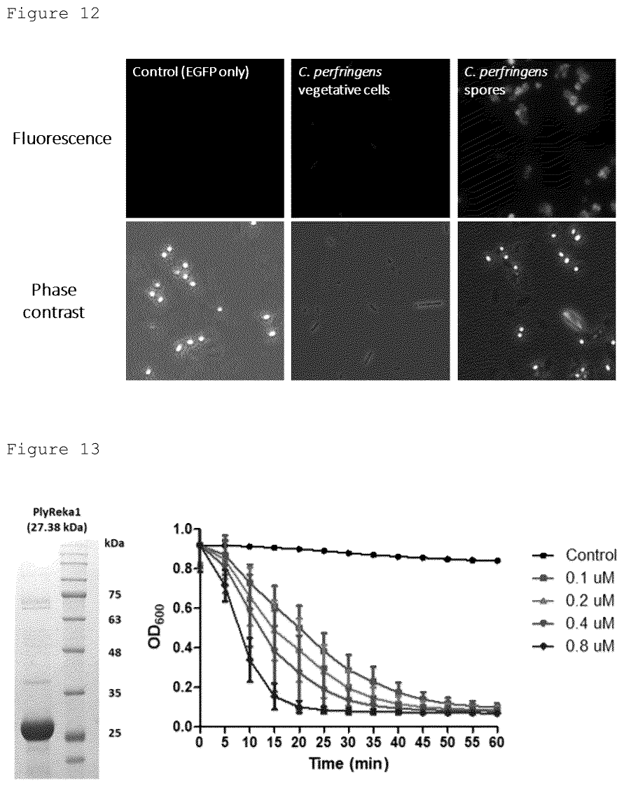

As a result of observation by fluorescence microscopy, it was confirmed that PlyReka1_SPOR bound only to the spores of Clostridium perfringens, but not to the vegetative cells of Clostridium perfringens and other Gram-positive bacteria or Gram-negative bacteria (FIG. 12). The C-terminus of PlyReka1 was named the spore-binding domain (SBD). The specificity of the present disclosure could be seen in that most Gram-positive phage endolysins have the CBD at the C-terminus, but PlyReka1 has the SBD, not the CBD.

### Example 6. Evaluation of Expression and Antibacterial Activity of PlyReka1 Protein

The protein was overexpressed and purified in a manner similar to Example 3. The putative endolysin region was amplified from the DNA of Reka1 by PCR and purified using the MiniBEST DNA fragment purification kit (Takara). The amplified product and pET28a were digested with BamHI and HindIII and ligated together, and the ligation product was transformed into E. coli. As a result, it was confirmed that the bacterial cells grew on medium containing kanamycin antibiotic, indicating that the recombinant plasmid was expressed normally in E. coli. Thereafter, a single colony of E. coli BL21 (DE3) was inoculated into the LB medium and grown by main culture at 37° C. and 250 rpm until the OD₆₀₀ value reached 0.6 to 0.8. Then, 0.5 mM IPTG was added thereto, followed by culturing at 18° C. and 250 rpm for 20 hours. The subsequent purification process thereafter was the same as in Example 3, and the concentration of the protein obtained by the above method was analyzed by Bradford assay, and the purity was analyzed by SDS-PAGE (FIG. 13).

### Example 7. Evaluation of Activity of ClyRD

A novel chimeric endolysin, ClyRD, was produced by linking amidase-3, an enzymatically active domain of PlyReka1, with the CBD of PlyDolk21, which has been confirmed to have excellent binding ability.

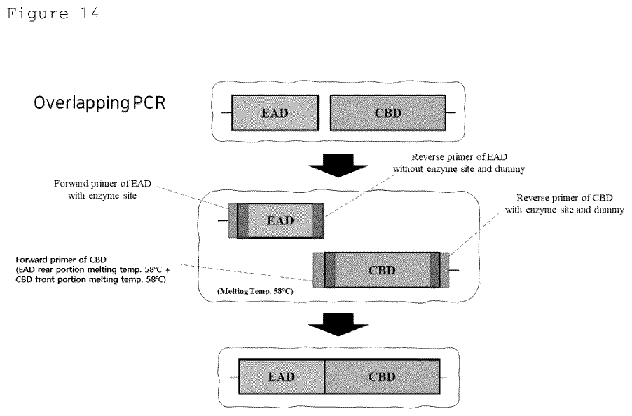

The EAD of PlyReka1 and the CBD of PlyDolk21 were each amplified by PCR using primers, and were then subjected to two rounds of PCR using primers designed for overlapping PCR, thereby artificially producing a chimeric endolysin (FIG. 14).

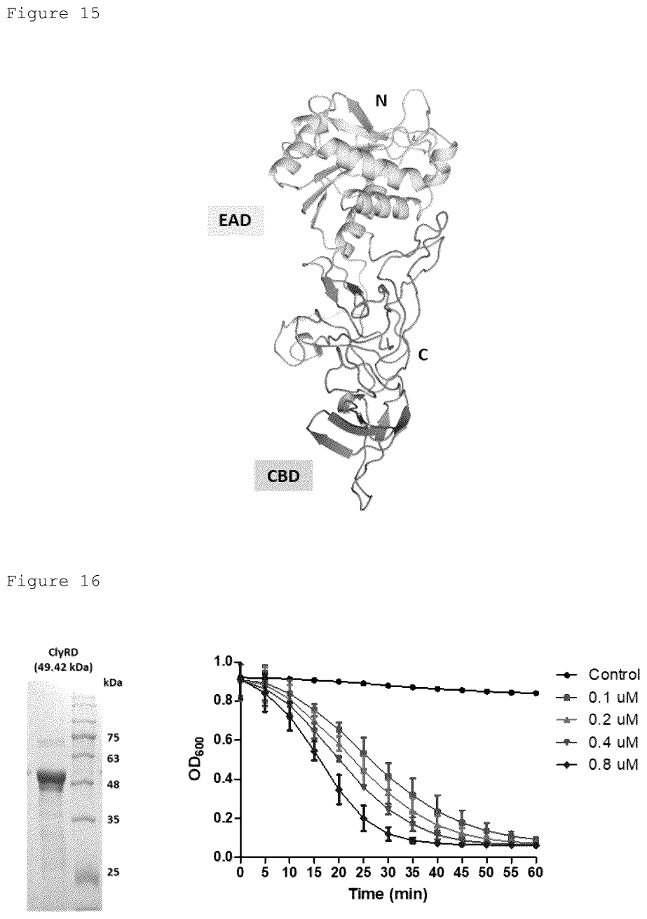

The 3D structure of ClyRD was predicted using Phyre2 (FIG. 15). The front and rear portions of ClyRD were treated with restriction enzymes BamHI and SalI, respectively. After the pET28a vector and the ClyRD protein were subjected to restriction enzyme treatment, they were ligated together. The protein expression and purification processes were performed in the same manner as in Example 6, and the activity of the ClyRD protein against Clostridium perfringens FD1 strain was analyzed by turbidity reduction assay (FIG. 16).

The EAD domain of ClyRD, which determines the activity of ClyRD, is derived from PlyReka1, and the results of comparing the antibacterial spectrum between the parent endolysin and the parent phage are shown in Table 2 below.

The lytic activity of the endolysin against Gram-positive and Gram-negative bacteria was confirmed based on a decrease in the OD600 value. The bacterial pellet in the early log phase was dissolved in reaction buffer (20 mM Tris-HCl, pH 8.0). The solution was dispensed into each well of a 24-well plate and treated with the endolysin according to each concentration to make 1 ml. Then, the absorbance of each well was measured using a microplate reader at 5-minute intervals for 40 minutes at 25° C. For comparison of the results, the values for 30 min were compared. To compare the spectrum of lytic activity, calculation of absorbance reduction was performed using Equation 1 below. (Observation for 30 minutes. −: 0 to 10%, +: 10 to 40%, ++: 40 to 70%, +++: 70 to 100%)

$$\begin{matrix}[ Equation 1 ] & \\ \{ \frac{( \vert O \text{?} of treatment - O \text{?} of control \text{?} )}{∈ ial O \text{?}} \times 100 ( \% ) \} . & Equation 1\end{matrix}$$

$$\text{?} \text{indicates text missing or illegible when filed}$$

TABLE 2
Antibacterial spectrum of phage and endolysin

| | | Bacteriophage | Endolysin | |
|---|---|---|---|---|
| Species | Strain | Reka1 | PlyReka1 | ClyRD |
| Gram-positive | | | | |
| C. perfringens | ATCC 13124 | + | +++ | +++ |
| | ATCC 3624 | − | +++ | +++ |
| | NCCP 15911 | − | +++ | ++ |
| | FD1 | − | +++ | +++ |
| | H3 | − | +++ | +++ |
| | H9 | − | +++ | +++ |
| | isolates 2589 | + | +++ | + |
| | isolates 2722 | − | + | + |
| Bacillus cereus | NCCP 10715 | − | − | − |
| B. subtilis | ATCC 23857 | − | − | − |
| B. amyloliquefaciens | KACC 15877 | − | − | − |
| Enterococcus faecalis | ATCC 10110 | − | − | − |
| Geobacillus | ATCC 10149 | − | − | − |
| stearothermophilus | | − | − | − |
| Listeria monocytogenes | ATCC 15313 | − | − | − |
| Staphylococcus aureus | Newman | − | − | − |
| Gram-negative | | | | |
| Escherichia coli | BL21 (DE3) | − | − | − |
| E. coli O157:H7 | OE50 | − | − | − |
| Pseudomonas putida | KCTC 1643 | − | − | − |
| Pseudomonas aeruginosa | ATCC 27853 | − | − | − |
| Yersinia enterocolitica | ATCC 55075 | − | − | − |
| Shigella flexneri | 2a strain 2457T | − | − | − |
| Salmonella Typhimurium | ATCC 43147 | − | − | − |
| Salmonella Enteritidis | ATCC 13076 | − | − | − |
| Cronobacter sakazakii | ATCC 29544 | − | − | − |

To determine the possibility of use in actual foods, the activity of the endolysin was evaluated in the milk and meat broth artificially contaminated with Clostridium perfringens. 1 ml of Clostridium perfringens ATCC 13124 (about 10⁷⁻⁸ CFU/ml) in the exponential phase was mixed with 1 ml of food. After dissolving the bacteria in the food at a ratio of 1/10 to make a total of 1 ml, the food was artificially inoculated with the bacteria through a pre-incubation process at 25° C. for 30 minutes. PlyReka1 and ClyRD were each inoculated into the sample at a predetermined concentration and incubated at 25° C. To count the number of viable cells 4 hours and 24 hours after endolysin treatment, 100 μl of the sample was diluted, spread on BHI medium and incubated overnight at 37° C.

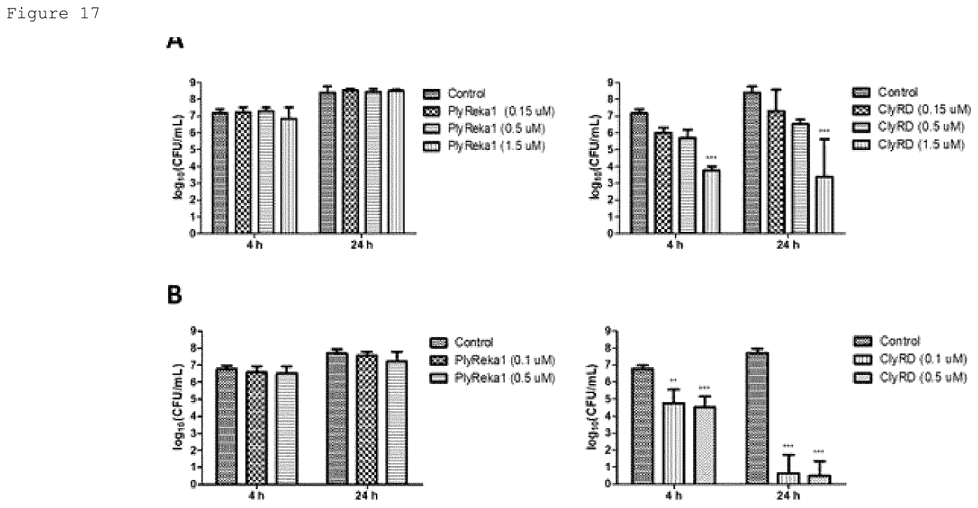

As a result, it was confirmed that, when the contaminated food was treated with each of the endolysins, PlyReka1 did not show activity, but when the food was treated with ClyRD, the number of viable cells decreased by 4.9-log CFU/mL and 7.3-log/CFU/mL, respectively, within 24 hours after treatment (FIG. 17).

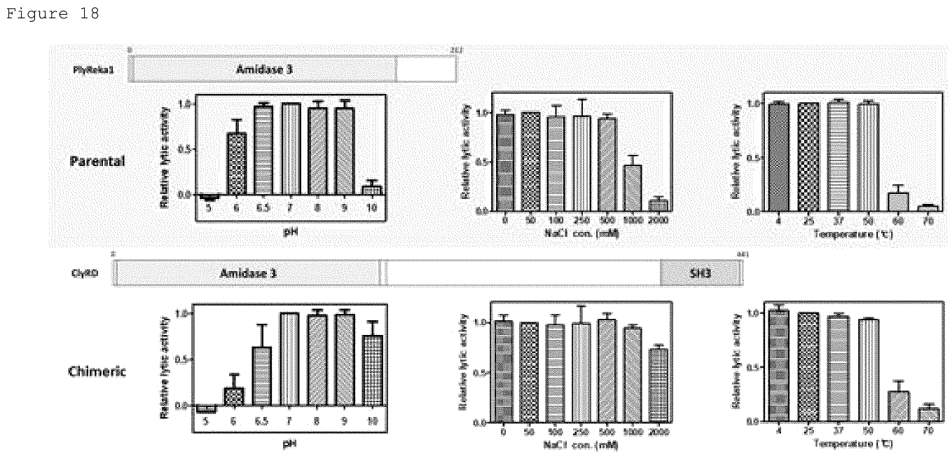

It was confirmed that the spectrum of lytic activity of ClyRD was identical to that of the parent endolysin PlyReka1, but the activity thereof was stable even at high pH and salt concentration (FIG. 18). These results suggest the novel chimeric endolysin ClyRD has the potential as a novel antibacterial agent for controlling Clostridium perfringens.

Table 3 below shows the DNA and amino acid sequences of the endolysins specified in the experimental examples above.

TABLE 3

| | |
|---|---|
| ClyRD DNA | atgaaaataggtattagagacggtcatagtccgaattgtaagggtgctataggtttac |
| sequence | gtgatgaacaatcatgtatgagagttttatgcaaagaagttatagaaatattagaaaa |
| (SEQ ID NO: 1) | acatggtcatgaagtagtttattgtggtagtgatgcaagtacacaaaatggtgaactt |
| | tcagaaggtgtgagaaaagctaataattcaaatgttgacatatttatttcactacaca |
| | tgaatagttttaacggacaagcccagggaacagaggcacttgttacagttggcgcaag |
| | aaactctataaaagaaattgcatcaaggttatgtaaaaactttgctagtttaggttta |
| | gtaaataggggtgtaaaagaagttaatttatatgaaatgaagaacgtaaaagcgccta |
| | acataatatttgaaactatgttttgtgataaccctcatgacataaacgaagtttggtc |
| | acctacaccatatgaaaaaatggctttactaattgcaaatgctatagacccaactatc |
| | aaaggagagagttcaagctcaccagtaattaataatcctaaaggattctatgagtcta |
| | atgagacaagaactaatgctactttagtgggggaaggttcaatagaagttctagatga |
| | ggattgtaagccagtccctggtagatttattgatagcctagatagtttatttgtcctt |
| | ggaatatatccttctagaaatttcatagaagttgtatatcctggaaaggataagaagt |
| | accatgcttacattgatataaaacattatagcagattaagttttgactaccacatgaa |
| | atatcaaaatgataatggaacaacttatgtgtggtggaaccctgaagacgttaatgtc |
| | aaagatcataatgaagaattacagccgggtcaaaaggctagcccaatgtatagaacta |
| | agggctggttaagaataacattctatagagaggatggaactccatcagatggatacgt |
| | tcgctacgagggtgagcaaagccagaaattctatgaggatgttaaacaaggaatagtt |
| | aaggttaacacttctcttaatgtaagagatgatgtgaatggtaatataataggctcag |
| | tatttagtaatgagaaggttactatattgggaagcaagaatggttggtatcatataga |
| | atataatactagccatggtaagaagcaaggttatgtaagttcaaaatatgtagagata |
| | atttag |
| ClyRD amino | MKIGIRDGHSPNCKGAIGLRDEQSCMRVLCKEVIEILEKHGHEVVYCGSDASTQNGEL |
| acid sequence | SEGVRKANNSNVDIFISLHMNSFNGQAQGTEALVTVGARNSIKEIASRLCKNFASLGL |
| (SEQ ID NO: 2) | VNRGVKEVNLYEMKNVKAPNIIFETMFCDNPHDINEVWSPTPYEKMALLIANAIDPTI |
| | KGESSSSPVINNPKGFYESNETRTNATLVGEGSIEVLDEDCKPVPGRFIDSLDSLFVL |
| | GIYPSRNFIEVVYPGKDKKYHAYIDIKHYSRLSFDYHMKYQNDNGTTYVWWNPEDVNV |
| | KDHNEELQPGQKASPMYRTKGWLRITFYREDGTPSDGYVRYEGEQSQKFYEDVKQGIV |
| | KVNTSLNVRDDVNGNIIGSVFSNEKVTILGSKNGWYHIEYNTSHGKKQGYVSSKYVEI |
| | I |
| PlyReka1 DNA | atgaaaataggtattagagacggtcatagtccgaattgtaagggtgctataggtttac |
| sequence | gtgatgaacaatcatgtatgagagttttatgcaaagaagttatagaaatattagaaaa |
| (SEQ ID NO: 3) | acatggtcatgaagtagtttattgtggtagtgatgcaagtacacaaaatggtgaactt |
| | tcagaaggtgtgagaaaagctaataattcaaatgttgacatatttatttcactacaca |
| | tgaatagttttaacggacaagcccagggaacagaggcacttgttacagttggcgcaag |
| | aaactctataaaagaaattgcatcaaggttatgtaaaaactttgctagtttaggttta |
| | gtaaataggggtgtaaaagaagttaatttatatgaaatgaagaacgtaaaagcgccta |
| | acataatatttgaaactatgttttgtgataaccctcatgacataaacgaagtttggtc |
| | acctacaccatatgaaaaaatggctttactaattgcaaatgctatagacccaactatc |
| | aaagaaaatgaactttatagagttgttgttcaatattttaacagcaaagaagatgctg |
| | aaaactgccaacaagaaatcgctaaaagatggtattgttttgtggaggaatgtaatta |
| | a |
| PlyReka1 | MKIGIRDGHSPNCKGAIGLRDEQSCMRVLCKEVIEILEKHGHEVVYCGSDASTQNGEL |
| amino acid | SEGVRKANNSNVDIFISLHMNSFNGQAQGTEALVTVGARNSIKEIASRLCKNFASLGL |
| sequence | VNRGVKEVNLYEMKNVKAPNIIFETMFCDNPHDINEVWSPTPYEKMALLIANAIDPTI |
| (SEQ ID NO: 4) | KENELYRVVVQYFNSKEDAENCQQEIAKRWYCFVEECN |
| PlyReka1_SPOR | ggatccgtaaaagcgcctaacataatatttgaaactatgttttgtgataaccctcatg |
| DNA sequence | acataaacgaagtttggtcacctacaccatatgaaaaaatggctttactaattgcaaa |
| (SEQ ID NO: 5) | tgctatagacccaactatcaaagaaaatgaactttatagagttgttgttcaatatttt |
| | aacagcaaagaagatgctgaaaactgccaacaagaaatcgctaaaagatggtattgtt |
| | ttgtggaggaatgtaattaa |
| PlyRekal_SPOR | VKAPNIIFETMFCDNPHDINEVWSPTPYEKMALLIANAIDPTIKENELYRVVVQYfNS |
| amino acid | KEDAENCQQEIAKRWYCFVEECN |
| sequence (SEQ | |
| ID NO: 6) | |
| EGFP:: | atggtgagcaagggcgaggagctgttcaccggggtggtgcccatcctggtcgagctgg |
| PlyReka1_SPOR | acggcgacgtaaacggccacaagttcagcgtgtccggcgagggcgagggcgatgccac |
| DNA sequence | ctacggcaagctgaccctgaagttcatctgcaccaccggcaagctgcccgtgccctgg |
| (SEQ ID NO: 7) | cccaccctcgtgaccaccctgacctacggcgtgcagtgcttcagccgctaccccgacc |
| | acatgaagcagcacgacttcttcaagtccgccatgcccgaaggctacgtccaggagcg |
| | caccatcttcttcaaggacgacggcaactacaagacccgcgccgaggtgaagttcgag |
| | ggcgacaccctggtgaaccgcatcgagctgaagggcatcgacttcaaggaggacggca |
| | acatcctggggcacaagctggagtacaactacaacagccacaacgtctatatcatggc |
| | cgacaagcagaagaacggcatcaaggtgaacttcaagatccgccacaacatcgaggac |
| | ggcagcgtgcagctcgccgaccactaccagcagaacacccccatcggcgacggccccg |
| | tgctgctgcccgacaaccactacctgagcacccagtccgccctgagcaaagaccccaa |
| | cgagaagcgcgatcacatggtcctgctggagttcgtgaccgccgccgggatcactctc |
| | ggcatggacgagctgtacaagggatccgtaaaagcgcctaacataatatttgaaacta |
| | tgttttgtgataaccctcatgacataaacgaagtttggtcacctacaccatatgaaaa |
| | aatggctttactaattgcaaatgctatagacccaactatcaaagaaaatgaactttat |
| | agagttgttgttcaatattttaacagcaaagaagatgctgaaaactgccaacaagaaa |
| | tcgctaaaagatggtattgttttgtggaggaatgtaattaa |
| EGFP:: | MVSKGEELFTGVVPILVELDGDVNGHKFSVSGEGEGDATYGKLTLKFICTTGKLPVPW |
| PlyReka1_SPOR | PTLVTTLTYGVQCFSRYPDHMKQHDFFKSAMPEGYVQERTIFFKDDGNYKTRAEVKFE |
| amino acid | GDTLVNRIELKGIDFKEDGNILGHKLEYNYNSHNVYIMADKQKNGIKVNFKIRHNIED |
| sequence | GSVQLADHYQQNTPIGDGPVLLPDNHYLSTQSALSKDPNEKRDHMVLLEFVTAAGITL |
| (SEQ ID NO: 8) | GMDELYKGSVKAPNIIFETMFCDNPHDINEVWSPTPYEKMALLIANAIDPTIKENELY |
| | RVVVQYFNSKEDAENCQQEIAKRWYCFVEECN |
| PlyDolk21 DNA | atgataatcaataaaagattaagtactactaatgttaccttaaacgctaataatccag |
| sequence | cctatataattatgcacgaaactgataacactgatagaggagcaggggctgaaagaca |
| (SEQ ID NO: 9) | ctgtagggctcaagctaatggaaatttaggagatgctagtgttcactattacgttgat |
| | gacaccggggtataccaagctgctgagcataaacacgctacttggaattgtggagatg |
| | gccataatagatatggtataaataatagaaatacaatatctatagaaatatgtgttaa |
| | tcctgactctgattataataaggcagttgataatgctgtagagctagttagataccta |
| | aaaaatggctactattctaattgtaaagtagtaagacactatgatgctagtagaaaaa |
| | attgtcctagaagaatgatagctaatggttactggaatacattcctagaaagagtaaa |
| | ttcaggagagagttcaagctcaccagtaattaataatcctaaaggattctatgagtct |
| | aatgagacaagaactaatgctactttagtgggggaaggttcaatagaagttctagatg |
| | aggattgtaagccagtccctggtagatttattgatagcctagatagtttatttgtcct |
| | tggaatatatccttctagaaatttcatagaagttgtatatcctggaaaggataagaag |
| | taccatgcttacattgatataaaacattatagcagattaagttttgactaccacatga |
| | aatatcaaaatgataatggaacaacttatgtgtggtggaaccctgaagacgttaatgt |
| | caaagatcataatgaagaattacagccgggtcaaaaggctagcccaatgtatagaact |
| | aagggctggttaagaataacattctatagagaggatggaactccatcagatggatacg |
| | ttcgctacgagggtgagcaaagccagaaattctatgaggatgttaaacaaggaatagt |
| | taaggttaacacttctcttaatgtaagagatgatgtgaatggtaatataataggctca |
| | gtatttagtaatgagaaggttactatattgggaagcaagaatggttggtatcatatag |
| | aatataatactagccatggtaagaagcaaggttatgtaagttcaaaatatgtagagat |
| | aatttag |
| PlyDolk21 | MIINKRLSTTNVTLNANNPAYIIMHETDNTDRGAGAERHCRAQANGNLGDASVHYYVD |
| amino acid | DTGVYQAAEHKHATWNCGDGHNRYGINNRNTISIEICVNPDSDYNKAVDNAVELVRYL |
| sequence | KNGYYSNCKVVRHYDASRKNCPRRMIANGYWNTFLERVNSGESSSSPVINNPKGFYES |
| (SEQ ID NO: 10) | NETRTNATLVGEGSIEVLDEDCKPVPGRFIDSLDSLFVLGIYPSRNFIEVVYPGKDKK |
| | YHAYIDIKHYSRLSFDYHMKYQNDNGTTYVWWNPEDVNVKDHNEELQPGQKASPMYRT |
| | KGWLRITFYREDGTPSDGYVRYEGEQSQKFYEDVKQGIVKVNTSLNVRDDVNGNIIGS |
| | VFSNEKVTILGSKNGWYHIEYNTSHGKKQGYVSSKYVEII |
| PlyDolk21_CBD | GGATCCggagagagttcaagctcaccagtaattaataatcctaaaggattctatgagt |
| DNA sequence | ctaatgagacaagaactaatgctactttagtgggggaaggttcaatagaagttctaga |
| (SEQ ID NO: 11) | tgaggattgtaagccagtccctggtagatttattgatagcctagatagtttatttgtc |
| | cttggaatatatccttctagaaatttcatagaagttgtatatcctggaaaggataaga |
| | agtaccatgcttacattgatataaaacattatagcagattaagttttgactaccacat |
| | gaaatatcaaaatgataatggaacaacttatgtgtggtggaaccctgaagacgttaat |
| | gtcaaagatcataatgaagaattacagccgggtcaaaaggctagcccaatgtatagaa |
| | ctaagggctggttaagaataacattctatagagaggatggaactccatcagatggata |
| | cgttcgctacgagggtgagcaaagccagaaattctatgaggatgttaaacaaggaata |
| | gttaaggttaacacttctcttaatgtaagagatgatgtgaatggtaatataataggct |
| | cagtatttagtaatgagaaggttactatattgggaagcaagaatggttggtatcatat |
| | agaatataatactagccatggtaagaagcaaggttatgtaagttcaaaatatgtagag |
| | ataatttag |
| PlyDolk21_CBD | GESSSSPVINNPKGFYESNETRTNATLVGEGSIEVLDEDCKPVPGRFIDSLDSLFVLG |
| amino acid | IYPSRNFIEVVYPGKDKKYHAYIDIKHYSRLSFDYHMKYQNDNGTTYVWWNPEDVNVK |
| sequence | DHNEELQPGQKASPMYRTKGWLRITFYREDGTPSDGYVRYEGEQSQKFYEDVKQGIVK |
| (SEQ ID NO: 12) | VNTSLNVRDDVNGNIIGSVFSNEKVTILGSKNGWYHIEYNTSHGKKQGYVSSKYVEII |
| EGFP:: | atggtgagcaagggcgaggagctgttcaccggggtggtgcccatcctggtcgagctgg |
| PlyDolk21_CBD | acggcgacgtaaacggccacaagttcagcgtgtccggcgagggcgagggcgatgccac |
| DNA sequence | ctacggcaagctgaccctgaagttcatctgcaccaccggcaagctgcccgtgccctgg |
| (SEQ ID NO: 13) | cccaccctcgtgaccaccctgacctacggcgtgcagtgcttcagccgctaccccgacc |
| | acatgaagcagcacgacttcttcaagtccgccatgcccgaaggctacgtccaggagcg |
| | caccatcttcttcaaggacgacggcaactacaagacccgcgccgaggtgaagttcgag |
| | ggcgacaccctggtgaaccgcatcgagctgaagggcatcgacttcaaggaggacggca |
| | acatcctggggcacaagctggagtacaactacaacagccacaacgtctatatcatggc |
| | cgacaagcagaagaacggcatcaaggtgaacttcaagatccgccacaacatcgaggac |
| | ggcagcgtgcagctcgccgaccactaccagcagaacacccccatcggcgacggccccg |
| | tgctgctgcccgacaaccactacctgagcacccagtccgccctgagcaaagaccccaa |
| | cgagaagcgcgatcacatggtcctgctggagttcgtgaccgccgccgggatcactctc |
| | ggcatggacgagctgtacaagggatccggagagagttcaagctcaccagtaattaata |
| | atcctaaaggattctatgagtctaatgagacaagaactaatgctactttagtggggga |
| | aggttcaatagaagttctagatgaggattgtaagccagtccctggtagatttattgat |
| | agcctagatagtttatttgtccttggaatatatccttctagaaatttcatagaagttg |
| | tatatcctggaaaggataagaagtaccatgcttacattgatataaaacattatagcag |
| | attaagttttgactaccacatgaaatatcaaaatgataatggaacaacttatgtgtgg |
| | tggaaccctgaagacgttaatgtcaaagatcataatgaagaattacagccgggtcaaa |
| | aggctagcccaatgtatagaactaagggctggttaagaataacattctatagagagga |
| | tggaactccatcagatggatacgttcgctacgagggtgagcaaagccagaaattctat |
| | gaggatgttaaacaaggaatagttaaggttaacacttctcttaatgtaagagatgatg |
| | tgaatggtaatataataggctcagtatttagtaatgagaaggttactatattgggaag |
| | caagaatggttggtatcatatagaatataatactagccatggtaagaagcaaggttat |
| | gtaagttcaaaatatgtagagataatttag |
| EGFP:: | MVSKGEELFTGVVPILVELDGDVNGHKFSVSGEGEGDATYGKLTLKFICTTGKLPVPW |
| PlyDolk21_CBD | PTLVTTLTYGVQCFSRYPDHMKQHDFFKSAMPEGYVQERTIFFKDDGNYKTRAEVKFE |
| amino acid | GDTLVNRIELKGIDFKEDGNILGHKLEYNYNSHNVYIMADKQKNGIKVNFKIRHNIED |
| sequence | GSVQLADHYQQNTPIGDGPVLLPDNHYLSTQSALSKDPNEKRDHMVLLEFVTAAGITL |
| (SEQ ID NO: 14) | GMDELYKGSGESSSSPVINNPKGFYESNETRTNATLVGEGSIEVLDEDCKPVPGRFID |
| | SLDSLFVLGIYPSRNFIEVVYPGKDKKYHAYIDIKHYSRLSFDYHMKYQNDNGTTYVW |
| | WNPEDVNVKDHNEELQPGQKASPMYRTKGWLRITFYREDGTPSDGYVRYEGEQSQKFY |
| | EDVKQGIVKVNTSLNVRDDVNGNIIGSVFSNEKVTILGSKNGWYHIEYNTSHGKKQGY |
| | VSSKYVEII |

So far, the present disclosure has been described with reference to the embodiments. Those of ordinary skill in the art to which the present disclosure pertains will appreciate that the present disclosure may be embodied in modified forms without departing from the essential characteristics of the present disclosure. Therefore, the disclosed embodiments should be considered from an illustrative point of view, not from a restrictive point of view. The scope of the present disclosure is defined by the claims rather than the foregoing description, and all differences within the scope equivalent thereto should be construed as being included in the present disclosure.

The present disclosure relates to discovering a more effective material for control and detection of Clostridium perfringens (C. perfringens) by isolating C. perfringens bacteriophages directly from an environmental sample and identifying and swapping several domains of a novel endolysin obtained through genomic analysis. The SBD obtained in the present disclosure may be used in various ways for detection and study of Clostridium perfringens spores, and the novel endolysin obtained by combining domains obtained from various endolysins demonstrates its superiority in that it can exhibit better activity and stability than the parent phage. Therefore, it was found that the present disclosure may be used as a probe for diagnosis and detection of live Clostridium perfringens and spores present in nature.

What is claimed is:

1. A chimeric endolysin protein comprising: an enzymatically active domain (EAD); a spore-binding domain (SBD); and a cell wall-binding domain (CBD).

2. The chimeric endolysin protein of claim 1, wherein the protein is one in which the enzymatically active domain, the spore-binding domain, and the cell wall-binding domain are sequentially linked together.

3. The chimeric endolysin protein of claim 1, wherein the enzymatically active domain, the spore-binding domain, and the cell wall-binding domain are derived from endolysin proteins of bacteriophages.

4. The chimeric endolysin protein of claim 1, wherein the protein has antibacterial activity against Clostridium sp. strains.

5. A protein for detecting foodborne disease-causing bacteria comprising the chimeric endolysin protein of claim 1.

6. The protein of claim 5, further comprising a fluorescent protein.

7. The protein of claim 5, wherein the detects of the clostridium sp. foodborne disease-causing bacteria are Clostridium sp. strains.

8. A composition for inhibiting foodborne disease-causing bacteria comprising the chimeric endolysin protein of claim 1.


SEQUENCE LISTING (14 sequences)

SEQ ID NO: 1 - DNA, 1224 bp, synthetic construct
  source 1..1224: mol_type = other DNA
atgaaaataggtattagagacggtcatagtccgaattgtaagggtgctataggtttacgtgatgaacaatcatgtatgagagttttatgcaaagaagttatagaaatattagaaaaacatggtcatgaagtagtttattgtggtagtgatgcaagtacacaaaatggtgaactttcagaaggtgtgagaaaagctaataattcaaatgttgacatatttatttcactacacatgaatagttttaacggacaagcccagggaacagaggcacttgttacagttggcgcaagaaactctataaaagaaattgcatcaaggttatgtaaaaactttgctagtttaggtttagtaaataggggtgtaaaagaagttaatttatatgaaatgaagaacgtaaaagcgcctaacataatatttgaaactatgttttgtgataaccctcatgacataaacgaagtttggtcacctacaccatatgaaaaaatggctttactaattgcaaatgctatagacccaactatcaaaggagagagttcaagctcaccagtaattaataatcctaaaggattctatgagtctaatgagacaagaactaatgctactttagtgggggaaggttcaatagaagttctagatgaggattgtaagccagtccctggtagatttattgatagcctagatagtttatttgtccttggaatatatccttctagaaatttcatagaagttgtatatcctggaaaggataagaagtaccatgcttacattgatataaaacattatagcagattaagttttgactaccacatgaaatatcaaaatgataatggaacaacttatgtgtggtggaaccctgaagacgttaatgtcaaagatcataatgaagaattacagccgggtcaaaaggctagcccaatgtatagaactaagggctggttaagaataacattctatagagaggatggaactccatcagatggatacgttcgctacgagggtgagcaaagccagaaattctatgaggatgttaaacaaggaatagttaaggttaacacttctcttaatgtaagagatgatgtgaatggtaatataataggctcagtatttagtaatgagaaggttactatattgggaagcaagaatggttggtatcatatagaatataatactagccatggtaagaagcaaggttatgtaagttcaaaatatgtagagataatttag

SEQ ID NO: 2 - protein, 407 aa, synthetic construct
MKIGIRDGHSPNCKGAIGLRDEQSCMRVLCKEVIEILEKHGHEVVYCGSDASTQNGELSEGVRKANNSNVDIFISLHMNSFNGQAQGTEALVTVGARNSIKEIASRLCKNFASLGLVNRGVKEVNLYEMKNVKAPNIIFETMFCDNPHDINEVWSPTPYEKMALLIANAIDPTIKGESSSSPVINNPKGFYESNETRTNATLVGEGSIEVLDEDCKPVPGRFIDSLDSLFVLGIYPSRNFIEVVYPGKDKKYHAYIDIKHYSRLSFDYHMKYQNDNGTTYVWWNPEDVNVKDHNEELQPGQKASPMYRTKGWLRITFYREDGTPSDGYVRYEGEQSQKFYEDVKQGIVKVNTSLNVRDDVNGNIIGSVFSNEKVTILGSKNGWYHIEYNTSHGKKQGYVSSKYVEII

SEQ ID NO: 3 - DNA, 639 bp, synthetic construct
  source 1..639: mol_type = other DNA
atgaaaataggtattagagacggtcatagtccgaattgtaagggtgctataggtttacgtgatgaacaatcatgtatgagagttttatgcaaagaagttatagaaatattagaaaaacatggtcatgaagtagtttattgtggtagtgatgcaagtacacaaaatggtgaactttcagaaggtgtgagaaaagctaataattcaaatgttgacatatttatttcactacacatgaatagttttaacggacaagcccagggaacagaggcacttgttacagttggcgcaagaaactctataaaagaaattgcatcaaggttatgtaaaaactttgctagtttaggtttagtaaataggggtgtaaaagaagttaatttatatgaaatgaagaacgtaaaagcgcctaacataatatttgaaactatgttttgtgataaccctcatgacataaacgaagtttggtcacctacaccatatgaaaaaatggctttactaattgcaaatgctatagacccaactatcaaagaaaatgaactttatagagttgttgttcaatattttaacagcaaagaagatgctgaaaactgccaacaagaaatcgctaaaagatggtattgttttgtggaggaatgtaattaa

SEQ ID NO: 4 - protein, 212 aa, synthetic construct
MKIGIRDGHSPNCKGAIGLRDEQSCMRVLCKEVIEILEKHGHEVVYCGSDASTQNGELSEGVRKANNSNVDIFISLHMNSFNGQAQGTEALVTVGARNSIKEIASRLCKNFASLGLVNRGVKEVNLYEMKNVKAPNIIFETMFCDNPHDINEVWSPTPYEKMALLIANAIDPTIKENELYRVVVQYFNSKEDAENCQQEIAKRWYCFVEECN

SEQ ID NO: 5 - DNA, 252 bp, synthetic construct
  source 1..252: mol_type = other DNA
ggatccgtaaaagcgcctaacataatatttgaaactatgttttgtgataaccctcatgacataaacgaagtttggtcacctacaccatatgaaaaaatggctttactaattgcaaatgctatagacccaactatcaaagaaaatgaactttatagagttgttgttcaatattttaacagcaaagaagatgctgaaaactgccaacaagaaatcgctaaaagatggtattgttttgtggaggaatgtaattaa

SEQ ID NO: 6 - protein, 82 aa, synthetic construct
SVKAPNIIFETMFCDNPHDINEVWSPTPYEKMALLIANAIDPTIKENELYRVVVQYFNSKEDAENCQQEIAKRWYCFVEECN

SEQ ID NO: 7 - DNA, 969 bp, synthetic construct
  source 1..969: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggatccgtaaaagcgcctaacataatatttgaaactatgttttgtgataaccctcatgacataaacgaagtttggtcacctacaccatatgaaaaaatggctttactaattgcaaatgctatagacccaactatcaaagaaaatgaactttatagagttgttgttcaatattttaacagcaaagaagatgctgaaaactgccaacaagaaatcgctaaaagatggtattgttttgtggaggaatgtaattaa

SEQ ID NO: 8 - protein, 322 aa, synthetic construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKGSVKAPNIIFETMFCDNPHDINEVWSPTPYEKMALLIANAIDPTIKENELYRVVVQYFNSKEDAENCQQEIAKRWYCFVEECN

SEQ ID NO: 9 - DNA, 1167 bp, synthetic construct
  source 1..1167: mol_type = other DNA
atgataatcaataaaagattaagtactactaatgttaccttaaacgctaataatccagcctatataattatgcacgaaactgataacactgatagaggagcaggggctgaaagacactgtagggctcaagctaatggaaatttaggagatgctagtgttcactattacgttgatgacaccggggtataccaagctgctgagcataaacacgctacttggaattgtggagatggccataatagatatggtataaataatagaaatacaatatctatagaaatatgtgttaatcctgactctgattataataaggcagttgataatgctgtagagctagttagatacctaaaaaatggctactattctaattgtaaagtagtaagacactatgatgctagtagaaaaaattgtcctagaagaatgatagctaatggttactggaatacattcctagaaagagtaaattcaggagagagttcaagctcaccagtaattaataatcctaaaggattctatgagtctaatgagacaagaactaatgctactttagtgggggaaggttcaatagaagttctagatgaggattgtaagccagtccctggtagatttattgatagcctagatagtttatttgtccttggaatatatccttctagaaatttcatagaagttgtatatcctggaaaggataagaagtaccatgcttacattgatataaaacattatagcagattaagttttgactaccacatgaaatatcaaaatgataatggaacaacttatgtgtggtggaaccctgaagacgttaatgtcaaagatcataatgaagaattacagccgggtcaaaaggctagcccaatgtatagaactaagggctggttaagaataacattctatagagaggatggaactccatcagatggatacgttcgctacgagggtgagcaaagccagaaattctatgaggatgttaaacaaggaatagttaaggttaacacttctcttaatgtaagagatgatgtgaatggtaatataataggctcagtatttagtaatgagaaggttactatattgggaagcaagaatggttggtatcatatagaatataatactagccatggtaagaagcaaggttatgtaagttcaaaatatgtagagataatttag

SEQ ID NO: 10 - protein, 388 aa, synthetic construct
MIINKRLSTTNVTLNANNPAYIIMHETDNTDRGAGAERHCRAQANGNLGDASVHYYVDDTGVYQAAEHKHATWNCGDGHNRYGINNRNTISIEICVNPDSDYNKAVDNAVELVRYLKNGYYSNCKVVRHYDASRKNCPRRMIANGYWNTFLERVNSGESSSSPVINNPKGFYESNETRTNATLVGEGSIEVLDEDCKPVPGRFIDSLDSLFVLGIYPSRNFIEVVYPGKDKKYHAYIDIKHYSRLSFDYHMKYQNDNGTTYVWWNPEDVNVKDHNEELQPGQKASPMYRTKGWLRITFYREDGTPSDGYVRYEGEQSQKFYEDVKQGIVKVNTSLNVRDDVNGNIIGSVFSNEKVTILGSKNGWYHIEYNTSHGKKQGYVSSKYVEII

SEQ ID NO: 11 - DNA, 705 bp, synthetic construct
  source 1..705: mol_type = other DNA
ggatccggagagagttcaagctcaccagtaattaataatcctaaaggattctatgagtctaatgagacaagaactaatgctactttagtgggggaaggttcaatagaagttctagatgaggattgtaagccagtccctggtagatttattgatagcctagatagtttatttgtccttggaatatatccttctagaaatttcatagaagttgtatatcctggaaaggataagaagtaccatgcttacattgatataaaacattatagcagattaagttttgactaccacatgaaatatcaaaatgataatggaacaacttatgtgtggtggaaccctgaagacgttaatgtcaaagatcataatgaagaattacagccgggtcaaaaggctagcccaatgtatagaactaagggctggttaagaataacattctatagagaggatggaactccatcagatggatacgttcgctacgagggtgagcaaagccagaaattctatgaggatgttaaacaaggaatagttaaggttaacacttctcttaatgtaagagatgatgtgaatggtaatataataggctcagtatttagtaatgagaaggttactatattgggaagcaagaatggttggtatcatatagaatataatactagccatggtaagaagcaaggttatgtaagttcaaaatatgtagagataatttag

SEQ ID NO: 12 - protein, 233 aa, synthetic construct
SGESSSSPVINNPKGFYESNETRTNATLVGEGSIEVLDEDCKPVPGRFIDSLDSLFVLGIYPSRNFIEVVYPGKDKKYHAYIDIKHYSRLSFDYHMKYQNDNGTTYVWWNPEDVNVKDHNEELQPGQKASPMYRTKGWLRITFYREDGTPSDGYVRYEGEQSQKFYEDVKQGIVKVNTSLNVRDDVNGNIIGSVFSNEKVTILGSKNGWYHIEYNTSHGKKQGYVSSKYVEII

SEQ ID NO: 13 - DNA, 1422 bp, synthetic construct
  source 1..1422: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggatccggagagagttcaagctcaccagtaattaataatcctaaaggattctatgagtctaatgagacaagaactaatgctactttagtgggggaaggttcaatagaagttctagatgaggattgtaagccagtccctggtagatttattgatagcctagatagtttatttgtccttggaatatatccttctagaaatttcatagaagttgtatatcctggaaaggataagaagtaccatgcttacattgatataaaacattatagcagattaagttttgactaccacatgaaatatcaaaatgataatggaacaacttatgtgtggtggaaccctgaagacgttaatgtcaaagatcataatgaagaattacagccgggtcaaaaggctagcccaatgtatagaactaagggctggttaagaataacattctatagagaggatggaactccatcagatggatacgttcgctacgagggtgagcaaagccagaaattctatgaggatgttaaacaaggaatagttaaggttaacacttctcttaatgtaagagatgatgtgaatggtaatataataggctcagtatttagtaatgagaaggttactatattgggaagcaagaatggttggtatcatatagaatataatactagccatggtaagaagcaaggttatgtaagttcaaaatatgtagagataatttag

SEQ ID NO: 14 - protein, 473 aa, synthetic construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKGSGESSSSPVINNPKGFYESNETRTNATLVGEGSIEVLDEDCKPVPGRFIDSLDSLFVLGIYPSRNFIEVVYPGKDKKYHAYIDIKHYSRLSFDYHMKYQNDNGTTYVWWNPEDVNVKDHNEELQPGQKASPMYRTKGWLRITFYREDGTPSDGYVRYEGEQSQKFYEDVKQGIVKVNTSLNVRDDVNGNIIGSVFSNEKVTILGSKNGWYHIEYNTSHGKKQGYVSSKYVEII